(19) United States
(12) Patent Application Publication
(10) Pub. No.: US 2005/0026998 A1
(43) Pub. Date: Feb. 3, 2005

(54) Compounds for the controlled release of active aldehydes

(76) Inventors: Gary B. Womack, Hopewell, NJ (US); Robert C. Vermeer, Columbus, NJ (US); Henry T. Kalinoski, Doylestown, PA (US)

Correspondence Address: WINSTON & STRAWN;PATENT DEPARTMENT, 1400 L STREET, N.W., WASHINGTON, DC, 20005-3502, US

(21) Appl. No.: 10/930,634

(22) Filed: Aug. 31, 2004

Related U.S. Application Data
(63) Continuation of application No. PCT/IB03/01198, filed on Mar. 27, 2003.
(60) Provisional application No. 60/369,115, filed on Mar. 28, 2002.

(51) Int. Cl. A61K 31/335

(52) U.S. Cl. 514/452

(57) ABSTRACT

The present invention relates to the field of perfumery. More particularly, it concerns aldoxane compounds of formula 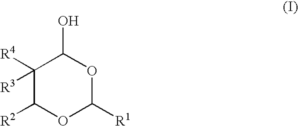
which compounds are capable of protecting an active aldehyde R¹CHO, for example, a perfumery or flavor aldehyde, from a chemically aggressive medium into which they have to be added, and then of releasing the active aldehyde at the desired moment. The present invention concerns also the use of such compounds in perfumery or in the flavor industry as well as the compositions or articles associated with these aldoxane compounds.

### CROSS-REFERENCE TO RELATED APPLICATIONS

This application is a continuation of International application PCT/IB03/01198 filed Mar. 27, 2003, which claims the benefit of provisional application 60/369,115 filed Mar. 28, 2002, the entire content of each of which is expressly incorporated herein by reference thereto.

### TECHNICAL FIELD

The present invention relates to the field of perfumery and flavorings. More particularly, it concerns aldoxane compounds capable of releasing an active aldehyde, for example a perfumery or flavor aldehyde, at the desired moment. The present invention concerns also the use of such compounds in the perfumery or in the flavor industry as well as the compositions or articles associated with these aldoxane compounds.

### BACKGROUND ART

Aldoxanes are a known family of compounds and, to the best of our knowledge, these compounds have never been described for a controlled release of an aldehyde capable of bringing a benefit or effect into its surrounding environment, e.g., a perfumery or flavor aldehyde.

Aldoxanes have been essentially reported as chemical intermediates, e.g., in the synthesis of surfactants. Of the aldoxanes responding to formula (I), described herein, and derived from an aldehyde which may be useful in perfumery or in the flavor industry, only the following have been reported in the prior art : 5-methyl-2,6-bis(1-methylethyl)-1,3-dioxan-4-ol, 2,6-diethyl-5-methyl-1,3-dioxan-4-ol, 2,6-dimethyl-1,3-dioxan-4-ol, 6-hexyl-2-(1-methylethyl)-1,3-dioxan-4-ol, 5,5,6-trimethyl-2-(1-methyl-ethyl)-1,3-dioxan-4-ol, 5,5-dimethyl-2,6-bis(1-methylethyl)-1,3-dioxan-4-ol, 2,6-dibenzyl-5-phenyl-1,3-dioxan-4-ol, 2-ethyl-6-methyl-1,3-dioxan-4-ol, 2-(1-methyl-ethenyl)-1,3-dioxan-4-ol, 6-hexyl-2-(2-octanol-1-yl)-1,3-dioxan-4-ol and 5-ethyl-2,6-dipropyl-1,3-dioxan-4-ol.

However none of these compounds has been described as being able to release an aldehyde in a controlled manner, nor their use as active ingredient in compositions such as perfuming or flavoring ones has been reported.

Additionally, 2-methyl-1,3-dioxan-4-ol has been reported as being a component of a muscat wine extract (A. Razungles et al. in Sci. Aliments, (1994) 14, 725-39), however the prior art is mute regarding its role as well as its behavior as an agent for the release of acetaldehyde.

### SUMMARY OF THE INVENTION

Surprisingly, we have now discovered that some aldoxane derivatives are capable of releasing an active aldehyde in a controlled manner, as well as of protecting these aldehydes, from a chemically aggressive medium into which it has to be added prior to its release or use. As “active aldehyde” we mean here any aldehyde capable of bringing a benefit or effect into its surrounding environment and in particular any aldehyde of current use in perfumery or in the flavor industry.

The aldoxane compounds of the invention are those of formula 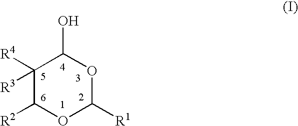
wherein R¹ represents an organic residue CY₃ derived from a perfuming or flavoring aldehyde of formula Y₃CCHO, Y being a hydrogen atom, a C₁ to C₂₀ linear, branched, cyclic or poly-cyclic saturated, unsaturated, aromatic or alkylaryl hydrocarbon radical, the hydrocarbon radical possibly comprising up to three oxygen or nitrogen atoms and being possibly substituted; two Y being possibly bonded together to form a saturated, unsaturated or aromatic ring having 5 to 20 carbon atoms, the ring optionally being substituted;
R² represents a R¹ group, a Y group or a C₅ to C₁₀ aromatic ring, the ring optionally possibly comprising up to three oxygen or nitrogen atoms and being possibly substituted; and
R³ and R⁴ represent each a Y group or are bonded together to form a saturated or unsaturated ring having 5 to 20 carbon atoms, the ring optionally being substituted.

The optional substituents are those which do not affect the function of the aldehyde and its release. Groups which are optional substituents of Y, R², R³, R⁴, and the rings which are possibly formed, are for example C₁ to C₈ linear, branched or cyclic alkyl or alkenyl groups, possibly containing one heteroatom such as an oxygen or nitrogen.

It is understood that whenever in a compound of formula (I) there are more than one Y group, then each the group may be identical to or different from the other Y groups. The same applies to R¹.

### DETAILED DESCRIPTION OF THE PREFERRED EMBODIMENTS

By the expression “perfuming or flavoring aldehyde” it is meant here a compound which is of current use in perfumery or in the flavor industry, i.e. a compound which is used as ingredient in flavoring or perfuming preparations or compositions in order to impart an hedonic effect. In other words, such an aldehyde, to be considered as being a perfuming or flavoring one, must be recognized by a person skilled in the art as being able to impart or modify in a positive and pleasant way the odor or taste of a composition, and not just as having an odor or taste.

Preferred compounds of formula (I) are those wherein R¹, R² and Y are defined as hereinabove; and R³ represents a C₁ to C₁₆ linear, branched, cyclic or poly-cyclic saturated, unsaturated, aromatic or alkylaryl hydrocarbon radical, the hydrocarbon radical optionally comprising up to three oxygen or nitrogen atoms and optionally being substituted; and
R⁴ represents a Y group or the R⁴ and R³ are bonded together to form a saturated or unsaturated ring having 5 to 15 carbon atoms, the ring optionally being substituted.

More preferred compounds of formula (I) are those wherein:
R¹ represents an organic residue derived from an active aldehyde of formula R¹CHO selected from the group consisting of hydroxycitronellal, citronellal, 3-(4-methoxyphenyl)-2-methylpropanal, the linear C₈ to C₁₂ alkyl aldehydes, 3-(4-isopropylphenyl)-2-methylpropanal, 3-(4-tert-butylphenyl)-2-methylpropanal, 4- and 3-(4-hydroxy-4-methylpentyl)-3-cyclohexene-1-carbaldehyde, 3-(4-tert-butylphenyl) propanal, 3-(1,3-benzodioxol-5-yl)-2-methylpropanal, 2,4-dimethyl-3-cyclohexene-1-carbaldehyde, 3- and 4-(3,3-dimethyl-5-indanyl)propanal, 8(9)-methoxy-tricyclo[5.2.1.0.(2,6)]decane-3(4)-carbaldehyde and 3-phenylbutanal;
R² represents a R¹ group, a hydrogen atom or a C₁ to C₁₆ linear, branched, cyclic or polycyclic alkyl, alkenyl or alkylaryl hydrocarbon radical, the hydrocarbon radical optionally being substituted, or a C₅ to C₆ aromatic ring, the ring optionally comprising up to three oxygen or nitrogen atoms and optionally being substituted;
R³ represents a C₁ to C₁₆ linear, branched, cyclic or poly-cyclic saturated, unsaturated, aromatic or alkylaryl hydrocarbon radical, the hydrocarbon radical optionally comprising up to three oxygen or nitrogen atoms and optionally being substituted; and
R⁴ represents a hydrogen atom or a R³ group; or R⁴ and R³ are bonded together to form a saturated or unsaturated ring having 5 to 10 carbon atoms, the ring optionally being substituted.

Yet more preferred are the compounds of formula (I) wherein R¹ is defined as hereinabove, R² and R³ represent a C₃ to C₁₀ linear, branched, cyclic or polycyclic saturated, unsaturated, aromatic or alkylaryl hydrocarbon radical, and R⁴ represents a hydrogen atom or a methyl or ethyl group.

The compounds of the invention can be synthesized by conventional methods using cheap starting materials. Generally speaking, the compounds of formula (I) are susceptible of being obtained by a reaction comprising the following step:
a) mixing together, the three aldehydes of the formulae (II), (III) and (IV), 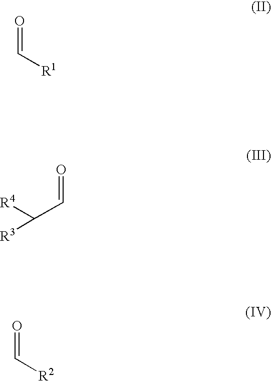
   wherein R¹, R², R³ and R⁴ have the meaning as defined in formula (I), in the presence of a base, such as an alkaline hydroxide or C₁ to C₄ alkoxide, and a temperature comprised between −10° C. and +10° C., preferably between 0° C. and 5° C., and aldehydes (III) and (IV) being in at least an equimolar amount in respect of aldehyde (II); or
b) reacting, at a temperature comprised between −10° C. and 50° C., preferably between 0° C. and 30° C., an active aldehyde (II), as defined hereinabove, with an aldol of formula 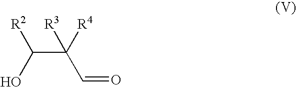
   wherein R², R³ and R⁴ have the meaning as defined in formula (I), the latter being susceptible of being obtainable by an aldol reaction between an aldehyde of formula (III) and an aldehyde of formula (IV).

The methods of synthesis of the aldol of formula (V) are well known to a person skilled in the art of the chemical synthesis.

General examples of this approach are illustrated in Scheme (I):
Scheme (I): Examples of Synthesis of the Compounds of Formula (I) 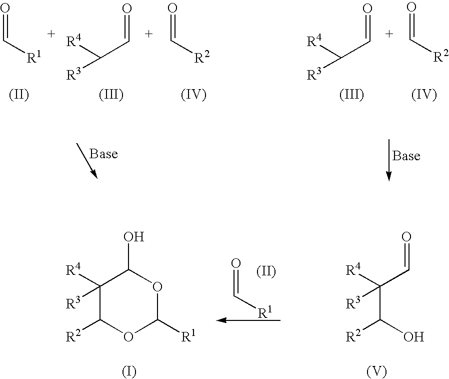
wherein the symbols R¹, R², R³ and R⁴ have the same meaning as defined in formula (I), and the base may be an alkaline hydroxide or C₁ to C₄ alkoxide.

General examples of such synthesis are described in the literature, e.g C. Chuit, et al. in Synthesis 1983, 294-296; R. H. Saunders, et al. in J. Am. Chem. Soc. 1943, vol. 65, 1714-1717; G. Fouquet, et al. in Liebigs Ann. Chem. 1979, 1591-1601; S. D. Rychnovsky, et al. in J. Org. Chem. 1992, vol. 57, 4336-4339; J. L. E. Erickson, et al. in J. Am. Chem. Soc., 1958, vol. 80, 5466-5469; Spaeth, et al. in Chem. Ber. 1943, vol. 76, 1196-1208 or Spaeth et al. in Chem. Ber. 1943, vol. 76, 513-520.

The aldehyde of formula (II) was previously defined as being an active aldehyde. The aldehydes of formulae (III) and (IV) are preferably identical, and may also be active aldehydes. Therefore it is possible to prepare an aldoxane of formula (I) using two or even three different active aldehydes. Whenever at least one of the aldehydes of the formulae (III) and (IV) is an active aldehyde, it is understood that they may be identical or different from the active aldehyde of formula (II).

An aldoxane of formula (I) susceptible of being obtained by the reaction of an active aldehyde of formula (II) with an aldol of formula (V), which is obtainable by the condensation of two identical or different aldehydes, is a preferred embodiment of the invention. In such a case, preferably, the aldol of formula (V) is obtained by the condensation of two C₃ to C₁₀ linear or branched aldehydes, more preferably pentanal or hexanal.

The compounds of the invention are capable of releasing an active aldehyde (II) via a decomposition reaction which is believed to be influenced by pH changes and/or heat, but may be triggered by other types of mechanisms. The decomposition reaction is illustrated in Scheme(II):
Scheme (II): Decomposition Reaction of the Compounds of Formula (I)

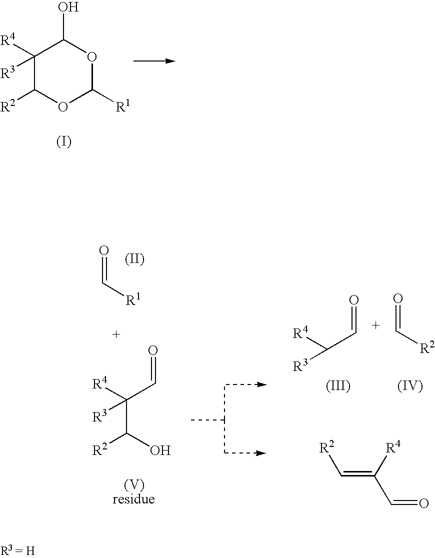

The decomposition reaction leads also to the release of the aldol (V) as a residue. It has to be pointed out that the residue may be itself a stable molecule or may decompose into an α,β-unsaturated aldehyde, via elimination of water, or into two molecules of aldehyde, via a retro-aldol reaction.

In the case the residue is a stable molecule, then preferably the residue is an inactive compound, e.g. an odorless aldol.

In the case the residue decomposes, then preferably the α,β-unsaturated aldehyde or the two molecules of aldehyde generated by the decomposition are active aldehydes, e.g. perfumery aldehydes. This embodiment of the invention is of particular interest as it allows, in principle, to achieve a “total mass efficiency”, meaning that no residue is generated, opposite to known aldehyde releasing systems such as classical 1,3-dioxanes.

The compounds of the invention are composed of two main parts, namely the aldol moiety, derived by the aldol of formula (V), and the active aldehyde moiety, derived by the active aldehyde of formula (II) and which is susceptible of being released.

Although it is not possible to provide an exhaustive list of the currently known active aldehyde of formula (II), the following perfumery or flavor aldehydes can be named as examples:

hydroxycitronellal, 3-(4-methoxyphenyl)-2-methylpropanal, 3,5,5-trimethylhexanal, 5- or 6-octenal, acetaldehyde, the linear C₆ to C₁₂ alkyl aldehydes and their α-methyl derivatives, hydratropic aldehyde, phenylacetaldehyde, 3-phenylpropanal, 3-(4-isopropylphenyl)propanal, 3-(4-methylphenyl)propanal, 4- or 6- or 8-nonenal, 9-decenal, 3,5-heptadienal, 3,5-nonadienal, 3,5-decadienal, 9-p-menthanal, PHENEXAL® [3-methyl-5-phenylpentanal, origin: Firmenich SA, Geneva, Switzerland], MUGOXAL® [3-(4-tert-butyl-1-cyclohexen-1-yl)propanal; origin: Firmenich SA, Geneva, Switzerland], 4-dodecenal, 4-decenal, 3,7-dimethyloctanal, citronellal, campholenic aldehyde, formyl pinane, LILIAL® [3-(4-tert-butylphenyl)-2-methylpropanal; origin: Givaudan-Roure SA, Vernier, Switzerland], LYRAL® [4- and 3-(4-hydroxy-4-methylpentyl)-3-cyclohexene-1-carbaldehyde; origin: International Flavors & Fragrances, USA], BOURGEONAL® [3-(4-tert-butylphenyl)propanal; origin: Quest International, Naarden, Netherlands], heliopropanal [3-(1,3-benzodioxol-5-yl)-2-methylpropanal; origin: Firmenich SA, Geneva, Switzerland], ZESTOVER (2,4-dimethyl-3-cyclohexene-1-carbaldehyde; origin: Firmenich SA, Geneva, Switzerland), TRIFERNAL® (3-phenylbutanal; origin: Firmenich SA, Geneva, Switzerland), (4-methylphenoxy)acetaldehyde, Scentenal® [8(9)-methoxy-tricyclo[5.2.1.0.(2,6)]decane-3(4)-carbaldehyde; origin: Firmenich SA, Geneva, Switzerland], LIMINAL® [(4R)-1-p-menthene-9-carbaldehyde; origin: Firmenich SA, Geneva, Switzerland], CYCLOSAL [3-(4-isopropylphenyl)-2-methylpropanal], 3-methyl-5-phenylpentanal, ACROPAL® [4-(4-methyl-3-pentenyl)-3-cyclohexene-1-carbaldehyde], 10-undecenal or 9-undecenal and their mixtures such as the INTERLEVEN® aldehyde (origin: International Flavors & Fragrances, USA), muguet aldehyde [(3,7-dimethyl-6-octenyl)acetaldehyde; origin: International Flavors & Fragrances, USA], 2,6-dimethyl-5-heptenal, PRECYCLEMONE®B [1-methyl-4-(4-methyl-3-pentenyl)-3-cyclohexen-1-carbaldehyde; origin: International Flavors & Fragrances, USA], HIVERNAL® [3 and 4-(3,3-dimethyl-5-indanyl)propanal; origin: Firmenich SA, Geneva, Switzerland] and ISOCYCLOCITRAL® (2,4,6-trimethyl-3-cyclohexene-1-carbaldehyde ; origin: International Flavors & Fragrances, USA).

The nature of the aldol moiety plays an important role in the release kinetics of the active aldehyde. Indeed, by changing the chemical nature of R², R³ and R⁴, e.g., the length or the branching of the groups, one can fine tune the perfume releasing properties of the aldoxane of the invention.

Furthermore, if the compounds of the invention are intended to be deposited on a surface, e.g. during a washing process, then the specific nature of R², R³ and R⁴ can also play an important role in the effective deposition and surface substantivity of the molecules of the invention on the surface used for the application, especially on fabrics and hair. For instance, sufficiently long and hydrophobic R², R³ and R⁴ groups increase substantially the substantivity of the aldoxane on the surface used for the application.

As previously, it is not possible to provide an exhaustive list of the aldehydes of formulae (III) and (IV) which can be used for the synthesis of the aldol (V) or of the aldoxane of the invention. However, for the aldehydes of formula (III), in addition to the cited aldehydes of formula (II), the following aldehydes can also be named as examples: acetaldehyde, propanaldehyde, butyraldehyde, isobutyraldehyde, pentanal, 3-methylpentanal, 2-methylpentanal, hexanal, heptanal, octanal, nonanal, decanal, dodecanal, 3-phenylpropanal and cyclohexanecarbaldehyde. For the aldehyde of formula (IV), in addition to the herein above cited compounds of formula (II) or (III), one can name as additional examples: formaldehyde, benzaldehyde, amyl or butyl cinnamic aldehyde, ortho or metha anisic aldehyde, cuminic aldehyde, 4-ethyl benzaldehyde, paratolueneic aldehyde, cinnamic aldehyde, 1,3-benzodioxol-5-carboxaldehyde, 2-tridecenal, 2,6,6-trimethyl-1,3-cyclohexadiene-1-carbaldehyde, citral, vanilline and ethyl vanilline.

We have already mentioned that the aldoxanes of the invention are particularly interesting for their ability to allow a controlled release of active aldehydes into the surrounding environment. We have also mentioned that another valuable advantage of the compounds is their ability to protect the active aldehyde from a chemically aggressive medium into which it has to be added. Yet another advantage of these compounds is that owing to their lower volatility, in respect to the free active aldehyde R¹CHO, they allow the use in application of highly volatile aldehydes, which are difficult to use because they do not persist. Therefore, the compound of formula (I), being a useful active ingredient, might be advantageously associated with compositions intended for applications such as the perfuming or the flavoring of various products.

In this respect, the present invention concerns also all different forms of the invention's compounds which can be advantageously employed in perfumery or in flavors. These forms are also embodiments of the present invention.

In one embodiment of the invention, one of these forms, which can be advantageously employed as perfuming or flavoring ingredient, is a composition of matter consisting of at least a compound of formula (I) and at least one perfumery or flavor carrier. By “perfumery or flavor carrier” we mean here one or more materials which are able to be admixed with an invention's compound without significantly altering its organoleptic properties, e.g. materials which are neutral from a perfumery or flavor point of view. The carrier may be a liquid or a solid.

As liquid carrier one may cite, as non-limiting examples, an emulsifying system, i.e. a solvent, e.g. water, and a surfactant system, or a solvent commonly used in perfumery or flavors. As examples of solvents commonly used in perfumery, generally speaking, one can cite compounds such as dipropyleneglycol, diethyl phthalate, isopropyl myristate, benzyl benzoate, 2-(2-ethoxyethoxy)-1-ethanol or ethyl citrate, which are the most commonly used. As examples of solvents commonly used in flavors, one can cite compounds such as propylene glycol, triacetine, triethyl citrate, benzylic alcohol, ethanol, vegetal oils or terpenes.

As solid carrier one may cite, as non-limiting examples, an absorbing gum or a polymer, or yet an encapsulating material. Such gums or materials are well known to a person skilled in the art and need no further explanation here.

In another embodiment of the invention, a suitable form of the compound (I) is a composition of matter comprising at least one compound of formula (I) and a perfume or flavor base. In other words the compound (I) is in the form of a perfuming or flavoring composition. It is understood that the perfuming ingredients are present in a perfuming effective amount.

Generally speaking, by “perfume or flavor base” we mean here a composition comprising at least one perfuming or flavoring co-ingredient and possibly one or more solvents and/or adjuvants commonly used in the perfume or flavor industry.

Such perfuming or flavoring co-ingredients are not of the formula (I) and may be in any of their forms. Moreover, by “perfuming or flavoring co-ingredient” it is also meant here a compound, which is of current use in perfumery or in the flavor industry, i.e. a compound which is used as ingredient in perfuming or flavoring preparation or composition in order to impart an hedonic effect. In other words such a co-ingredient, to be considered as being a perfuming or flavoring one, must be recognized by a person skilled in the art as being able to impart or modify in a positive or pleasant way the odor or taste of a composition, and not just as having an odor or taste. Its is therefore understood here that, unless otherwise indicated or described, any mixture resulting from a chemical synthesis in which the compound of the invention is involved as a starting intermediate or as an end-product is not a perfuming or flavoring base according to the invention.

The nature and type of the perfuming or flavoring co-ingredients present in the base do not warrant a more detailed description here, which in any case would not be exhaustive, the skilled person being able to select them on the basis of its general knowledge and according to intended use or application and the desired organoleptic effect. In general terms, these perfuming co-ingredients belong to chemical classes as varied as alcohols, aldehydes, ketones, esters, ethers, acetates, nitrites, terpene hydrocarbons, nitrogenous or sulphurous heterocyclic compounds and essential oils of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery or flavors. It is also understood that the co-ingredients may also be compounds known to release in a controlled manner various types of perfuming or flavoring compounds.

Similarly, a detailed description of the nature and type of solvents commonly used in perfuming or flavoring bases cannot be exhaustive. A skilled person in the art is able to select them on the basis of the nature of the product to be perfumed. However, as non-limiting examples of solvents commonly used in perfumery bases, one can cite, in addition to the solvents mentioned above, also ethanol, water/ethanol mixtures, limonene or other terpenes, isoparaffins such as those known under the trademark ISOPAR® (origin: Exxon Chemical) or glycol ethers and glycol ether esters such as those known under the trademark DOWANOL® (origin: Dow Chemical Company). As non-limiting examples of solvents commonly used in flavoring bases, one can cite, the same as listed above.

The perfuming or flavoring compositions according to the invention may be a simple mixture of the various co-ingredients and solvents, or also in the form of a bi-phasic system such as an emulsion or microemulsion. Such systems are well known to a person skilled in the art. In the case of flavoring bases, the latter may also be a simple mixture of flavoring ingredients or also in an encapsulated form as mentioned above.

The possibility to have, in the compositions of matter mentioned above, more than one compound of formula (I) is important as it enables the perfumer or flavorist to prepare accords, perfumes or flavors, possessing the ability to release in a controlled manner more than one perfuming or flavoring aldehyde.

One problem of perfuming ingredients, in any of their forms, present as such in washing compositions is that they can have little staying-power and be consequently often eliminated in the rinsing water or upon drying of surface. Another problem is that the perfuming ingredients, once incorporated into a washing compositions, can also be unstable and be transformed into odor less or bad odorant compounds. These problems can be solved by using a compound of formula (I), for which we have been able to show, on the one hand, that they possess a surprising stability over storage and staying-power or tenacity on surfaces, especially on textiles, and on the other hand, they may also play the role of “stabilizer” allowing thus the use of chemically fragile or highly volatile aldehydes.

Therefore, the aldoxanes of formula (I), owing to their properties can be incorporated in any application requiring the effect of rapid or prolonged liberation of a fragrant aldehyde as defined hereinabove. In particular, they can be used in functional or fine perfumery, particularly in applications in which the fragrance and the freshness of the ingredients must be effectively imparted to the treated surface during washing well beyond the rinsing and drying processes. Suitable surfaces are, in particular, textiles, hard surface, hair and skin.

One of the chief advantages of the invention resides in the fact that the compounds impart an intense fragrance to the treated surface, produced by a fragrant aldehyde, which would not be detected on the surface over a sufficiently long period if the fragrant aldehyde had been used as it is, i.e., without a precursor.

Therefore, a compound of formula (I), in any of its forms, is a useful perfuming ingredient which can be advantageously used in all the fields of modern perfumery, such as fine perfumery or functional perfumery, to positively impart or modify the odor of a consumer product into which the compound (I) is added.

Consequently, a perfumed article comprising:
i) at least one compound of formula (I), or any of its forms mentioned above; and
ii) a consumer product base,
is also an embodiment of the present invention.

For the sake of clarity, it has to be mentioned that, by “consumer product base” we mean here an unperfumed consumer product, i.e., a consumable product such as a detergent or a perfume, or a part of the consumer product. Therefore, a perfumed article according to the invention comprises at least a part of the whole formulation corresponding to a desired consumer product, e.g. a detergent, and an olfactive effective amount of at least an invention's compound, possibly together with one or more perfuming co-ingredient, solvent and/or adjuvant.

Suitable unperfumed consumer products comprise solid or liquid detergents and fabric softeners as well as all the other articles common in perfumery, namely perfumes, colognes or after-shave lotions, perfumed soaps, shower or bath salts, mousses, oils or gels, hygiene products or hair care products such as shampoos, body-care products, deodorants or antiperspirants, air fresheners and also cosmetic preparations. As detergents are intended applications such as detergent compositions or cleaning products for washing up or for cleaning various surfaces, e.g., intended for textile, dish or hard-surface treatment, whether they are intended for domestic or industrial use. Other perfumed articles are fabric refreshers, ironing waters, papers, wipes or bleaches.

Preferred unperfumed consumer products are fabric detergents or softener bases.

Some of the above-mentioned consumer product bases may represent an aggressive medium for the invention compounds, so that it may be necessary to protect the latter from premature decomposition, for example by encapsulation.

The nature and type of the constituents of the consumer product do not warrant a more detailed description here, which in any case would not be exhaustive, the skilled person being able to select them on the basis of its general knowledge and according to the nature and the desired effect of the product. However, as typical examples of fabric detergents or softener compositions into which the compounds of the invention can be incorporated, one may cite the one described in WO 97/34986 or in U.S. Pat. Nos. 4,137,180 and 5,236,615 or EP 799 885. Other typical detergent and softening compositions which can be used are described in works such as Ullman's Encyclopedia of Industrial Chemistry, vol. A8, pages 315-448 (1987) and vol. A25, pages 747-817 (1994); Flick, Advanced Cleaning Product Formulations, Noye Publication, Park Ridge, N.J. (1989); Showell, in Surfactant Science Series, vol. 71: Powdered Detergents, Marcel Dekker, New York (1988); Proceedings of the World Conference on Detergents (4th, 1998, Montreux, Switzerland), AOCS print.

The proportions in which the compounds according to the invention can be incorporated into the various aforementioned products vary within a wide range of values. These values are dependent on the nature of the article or product to be perfumed and on the desired olfactory effect as well as the nature of the co-ingredients in a given composition when the compounds according to the invention are mixed with perfuming co-ingredients, solvents or additives commonly used in the art.

For example, typical concentrations are in the order of 0.01% to 50% by weight, or even more, of these compounds based on the weight of the composition into which they are incorporated. Concentrations in the order of 0.001% to 5% by weight can be used when these compounds are applied directly in the perfuming of the various consumer products mentioned hereinabove.

As previously mentioned, a compound of formula (I) is also a useful flavoring ingredient which can be advantageously incorporated into flavored article to positively impart, or modify, the taste of the article. Consequently, a flavored article comprising:
i) at least one compound of formula (I), or any of its forms above-mentioned; and
ii) a foodstuff base,
is also an object of the present invention.

Suitable foodstuffs, e.g. foods or beverages, include products such as such as, for examples, dry powder or concentrated compositions for instant beverages, such as fruit juices or hot soups, chewing gum and baking applications such as cake mixes or cookie dough.

The nature and type of the constituents of the foodstuffs or beverages do not warrant a more detailed description here, which in any case would not be exhaustive, the skilled person being able to select them on the basis of its general knowledge and according to the nature of the product.

The present invention relates also about the use of an invention's compound as perfuming or flavoring ingredient. In other words it concerns a method to confer, enhance, improve or modify the odor or flavor properties of a perfuming or flavoring composition or of a perfumed or flavored article, which method comprises adding to the composition or article an effective amount of at least a compound of formula (I). By “use of a compound of formula (I)” it has to be understood here also the use of the compound (I) in any of its forms which can be advantageously employed in perfumery or in flavors as active ingredients. Another object of the invention is the use of a compound of formula (I) as a precursor capable of liberating an active aldehyde, e.g. a perfuming aldehyde, or a mixture thereof. Such uses are particularly attractive when they occur in an aggressive medium, i.e. a medium in which the active aldehyde as such will be chemically unstable.

Yet another embodiment of the present invention relates to a process for the perfuming of a surface or a process for intensifying, prolonging or deferring the diffusion effect of the characteristic fragrance of a fragrant aldehyde on a surface, characterized in that the surface is treated in the presence of a compound of formula (I), as defined above. Suitable surfaces are, in particular, textiles, hard surfaces, hair and skin. Preferably, the compound of formula (I) is comprised in an appropriate composition or article cited above.

An additional embodiment of the invention relates to a method for perfuming a surface or object which comprises adding a composition described herein, which composition includes a perfume base comprising an active aldehyde, to a chemically aggressive medium, and then releasing the active aldehyde at the desired moment, wherein the composition protects the aldehyde from degradation due to exposure to the chemically agressive medium.

Furthermore, the invention relates to a method for flavoring an edible substance which comprises adding a composition described herein, which composition includes a flavor base comprising an active aldehyde, to a chemically aggressive medium, and then releasing the active aldehyde at the desired moment, wherein the composition protects the aldehyde from degradation due to exposure to the chemically agressive medium.

### EXAMPLES

The invention will now be described in further details by way of the following examples, wherein the abbreviations have the usual meaning in the art, the temperatures are indicated in degrees centigrade (° C.); ₁H-NMR spectral data were recorded at 360 MHz and ¹³C NMR spectra were recorded at 90 MHz in CDCl₃, the chemical displacement δ are indicated in ppm with respect to the TMS as standard, the coupling constants J are expressed in Hz. Fractional distillations were performed with a 10 cm Vigreux column. All GC analysis were performed on the acetates derivatives of the final products. The acetates derivatives were prepared by mixing the sample with an acetylating reagent at a 1:4 volume ratio in a GC vial; the acetylating reagent was prepared by mixing 1 ml each of acetic anhydride and pyridine and 50 mg of 4-(dimethylamino)pyridine (DMAP). GC-FID analyses were performed with a 30 m capillary column (ID 0.32 mm) coated with 5%-diphenyl-95%-dimethylsiloxane copolymer (0.32 um film thickness). GC-MS analyses were conducted using either a Hewlett-Packard 5989A or HP 6890 mass spectrometer operated with an ionization energy of 70 eV and a mass detection range sufficient to detect the molecular ion of the compound of interest. All aldoxane MS data was obtained using the acetate derivatives.

### Example 1

Preparation of Aldoxanes by the Self-reaction of Three Identical Aldehydes

General KOH-Catalyzed Procedure

An aqueous 10% KOH solution (10-15 mole % KOH on the aldehyde) was cooled to 0° C. The aldehyde was added dropwise at a rate to keep the temperature of the reaction mixture below 5° C. The reaction mixtures were stirred with an overhead stirrer, generally 15-20 hours, while being maintained at or below 5° C. In the case the reaction medium solidified diethyl ether was added to these mixtures to keep it liquid. While still cold, the aqueous and organic phases of the reaction mixtures were separated. The aqueous phase was extracted with diethyl ether. The combined organic phases were washed with water until the aqueous phase was neutral. The organic phases were dried over Na₂SO₄, filtered and concentrated on a rotary evaporator. Residual solvent was further removed under vacuum to yield the aldoxane in 90-99% yield. Using this procedure, aldoxanes were obtained using butanal (a), isobutanal (b), pentanal (c), isovaleraldehyde (d), hexanal (e), octanal (f), decanal (g), phenylacetaldehyde (h), 3-phenylpropanal (i), and 2-phenylpropanal (j), heptanal (k) and 9-undecenal (l). The aldoxanes obtained from butanal, isobutanal, pentanal, hexanal, heptanal and 9-undecenal were liquid at ambient temperature, all the others were solids or semi-solids. GC-FID and GC-MS analyses showed that the mixtures were composed predominantly of the desired aldoxane. Mass spectral data is reported for the most prominent aldoxane isomer. IR spectroscopy also confirmed the aldoxane structure, showing a strong O-H absorption band and only a weak carbonyl absorption, if any, and strong bands in the C-O stretching region.

a. 2,6-dipropyl-5-ethyl-1,3-dioxan-4-ol (obtained in 94% yield)

MS: 258 (M⁺, <1), 257 (M⁺-1, 1), 199 (M⁺-MeCO₂, 3), 215 (4), 169 (11), 127 (26), 114 (50), 98 (26), 72 (33), 43 (100).

b. 2,6-diisopropyl-5,5-dimethyl-1,3-dioxan-4-ol (obtained in 97% yield)

IR (film): 3431 (s broad), 2966 (s), 2878 (m) 1720 (w), 1472 (m), 1407 (w), 1385 (w), 1367 (w), 1295 (w), 1195 (w), 1106 (s broad), 1024 (s), 998 (m), 956 (m) 887 (w), 788 (w) cm⁻¹.

MS: 258 (M⁺, <1), 257 (M⁺-1, 1), 199 (M⁺-MeCO₂, 1), 215 (5), 169 (3), 127 (18), 114 (58), 98 (39), 72 (65), 43 (100).

c. 2,6-dibutyl-5-propyl-1,3-dioxan-4-ol (obtained in 96% yield)

IR (film): 3417 (s broad), 2959 (s), 2936 (s), 2874 (s), 1466 (m), 1380 (m), 1148 (s), 1108 (sh), 991 (m), 965 (m) cm⁻¹.

MS: 300 (M⁺, <1), 299 (M⁺-1, 1), 243 (3), 241 (M⁺-MeCO₂, 2), 197 (5), 155 (19), 128 (30), 126 (25), 86 (34), 43 (100).

d. 2,6-di(2-methylpropyl)-5-isopropyl-1,3-dioxan-4-ol (obtained in 91% yield)

IR (film): 3412 (m broad), 2958 (s), 2938 (sh), 2873 (m), 1721 (w), 1469 (m), 1385 (w), 1368 (m), 1149 (m), 1120 (m), 1052 (w), 991 (m), 963 (m) cm⁻¹.

MS: 300 (M⁺, <1), 299 (M⁺-1, 1), 243 (3), 241 (M⁺-MeCO₂, 2), 197 (4), 155 (8), 137 (15),128 (15), 126 (9), 86 (34), 68 (46), 43 (100).

e. 2,6-dipentyl-5-butyl-1,3-dioxan-4-ol (obtained in 96% yield)

IR (film): 3417 (m broad), 2957 (s), 2932 (s), 2862 (m), 1723 (w), 1465 (m), 1417 (w), 1379 (m), 1341 (w), 1147 (m), 1110 (sh), 986 (m), 958 (m) cm⁻¹.

MS: 342 (M⁺, 1), 341 (M⁺-1, 5), 283 (M⁺-MeCO₂, 12), 271 (29), 225 (31), 183 (58), 154 (76), 142 (50), 100 (43), 82 (74), 43 (100).

f. 2,6-diheptyl-5-hexyl-1,3-dioxan-4-ol (obtained in 96% yield)

IR (film): 3416 (m broad), 2956 (s), 2927 (s), 2858 (s), 1724 (w), 1465 (m), 1378 (m), 1146 (m), 1115 (w), 1063 (w), 967 (m) cm⁻¹.

MS: 426 (M⁺, <1), 425 (M⁺-1, 1), 367 (M⁺-MeCO₂, 2), 327 (7), 281 (12), 239 (27), 210 (15), 170 (28), 128 (16), 110 (67), 43 (100).

g. 2,6-dinonyl-5-octyl-1,3-dioxan-4-ol (obtained in 92% yield)

IR (film): 3414 (m broad), 2955 (m), 2925 (m), 2855 (m), 1726 (w), 1693 (w), 1465 (m), 1377 (w), 1144 (m), 1117 (w), 983 (m broad) cm⁻¹.

MS: 451 (M⁺-MeCO₂, 5), 337 (8), 296 (15), 155 (44), 57 (100), 43 (84).

h. 2,6-dibenzyl-5-phenyl-1,3-dioxan-4-ol (obtained in 90% yield)

IR (film): 3394(s broad), 3062 (w), 3029 (m), 2976 (m), 2916 (m), 1603 (w), 1496 (m), 1454 (m), 1105 (s), 1079 (s), 1043 (s) cm⁻¹.

MS: 384 (M⁺-18, 26), 342 (M⁺-60, <1), 324 (10), 264 (26), 233 (86), 222 (100), 91 (71), 43 (49).

i. 2,6-di(2-phenylethyl)-5-benzyl-1,3-dioxan-4-ol (obtained in 97% yield)

IR (film): 3420 (m broad), 3085 (w), 3062 (w), 3027 (m), 2931 (m), 2865 (m), 1603 (w), 1496 (m), 1454 (m), 1137 (s), 1048 (m), 948 (w) cm⁻¹.

MS: 444 (M⁺, <1), 384 (M⁺-60, 2), 275 (9), 159 (12), 145 (14), 133 (23), 131 (23), 117 (31), 91 (100), 43 (19).

j. 2,6-di(1-phenylethyl)-5-methyl-5-phenyl-1,3-dioxan-4-ol (obtained in 91% yield)

The reaction mixture obtained with hydratropic aldehyde (2-phenylpropanal) was approximately a 50/50 mix of the aldehyde and aldoxane.

IR(film): 3440 (m broad), 2978 (s), 1720 (s), 1495 (s), 1453 (s), 1145 (s), 1102 (s), 1068 (s), 1020 (s) cm⁻¹.

MS: 443 (M⁺-1, <1), 222 (48), 207(43), 176 (41), 134 (100), 105 (67), 43 (35).

k. 2,6-di(hexyl)-5-pentyl-1,3-dioxan-4-ol (obtained in 95% yield)

IR (film): 3415 (m broad), 2956 (s), 2930 (s), 2859 (s), 1725 (w), 1465 (m), 1417 (w), 1378 (m), 1147 (m), 1113 (m), 1057 (w), 957 (m) cm⁻¹.

MS: 384 (M⁺, <1), 383 (M⁺-1, 1), 325 (M⁺-MeCO₂, 2), 299 (5), 253 (13), 211 (29), 182(18), 156 (33), 96 (83), 43 (100).

l. 2,6-di(dec-8-en-1-yl)-5-(non-7-en-1-yl)-1,3-dioxan-4-ol (obtained in 83% yield)

IR (film): 3413 (m broad), 3015 (m), 2927 (vs), 2855 (s), 1727 (w), 1657 (vw), 1642 (vw), 1461 (m), 1441 (m), 1372 (w) 1142 (s), 965 (s) cm⁻¹.

### Example 2

Preparation of Aldoxanes by the Self-reaction of Two Different Aldehydes

a) 2-Pentyl-5-butyl-6-phenyl-1,3-dioxan-4-ol

Benzaldehyde (5 grams, 0.047 moles) and methanol (5 ml) were added to a flask, and the mixture cooled with a 0° C. cold bath. Sodium methoxide (1 gram, 25% methanolic solution, 4.6 mmoles) was added, followed by the dropwise addition of 9.4 grams of hexanal. The reaction mixture was stirred for four hours. Diethyl ether (100 ml) and water (25 ml) were added and the water phase was washed with 50 ml of diethyl ether. The ether phases were combined and washed with water (3×50 ml) until the aqueous phase was neutral. The ether phase was dried over Na₂SO₄, filtered and concentrated on a rotary evaporator yielding 12.3 g (85% yield) of a colorless, viscous liquid. GC-MS analysis showed the major component was 2-pentyl-5-butyl-6-phenyl-1,3-dioxan-4-ol (at least two isomers). Also found in the product were benzaldehyde, hexanal, the aldoxane of hexanal (see example 1e) and the 2-butylcinnamic aldehyde.

IR (film): 3418 (s, broad), 2957 (s), 2932 (s), 2862 (s), 1705 (w), 1457 (m), 1140 (s) cm⁻¹.

MS: 347 (M⁺-1, <1), 289 (M⁺-MeCO₂, 1), 248 (8), 189 (36), 160 (15), 142 (38), 91 (82), 82 (55), 43 (100).

b) 2,6-Dipentyl-5-methyl-5-phenyl-1,3-dioxan-4-ol

A mixture of hydratropic aldehyde (13.4 grams, 0.1 moles), hexanal (20 grams, 0.2 moles) and diethyl ether (50 ml) was added dropwise to 11 ml of a cold (0° C.) 10% KOH solution. The mixture was stirred an additional 15 hours at 0° C. The mixture was added to a separatory funnel and the aqueous phase removed. The organic phase was washed with water until the pH of the aqueous phase was neutral. The ether phase was dried over Na₂SO₄, filtered and concentrated on a rotary evaporator yielding 28.4 g (85% yield) of a colorless, viscous liquid. GC-MS analysis showed the major component was 2,6-dipentyl-5-methyl-5-phenyl-1,3-dioxan-4-ol. The isomer formed from the hexanal aldol adding to hydratropic aldehyde, 2-(1-phenylethyl)-5-butyl-6-pentyl-1,3-dioxan-4-ol (example 5.5.12), could only be found by extracted ion analysis using ions 183 and 225 of the total ion chromatogram. It was present at a very low level relative to the major product.

Detected in minor amounts were the aldoxane of hexanal (see example 1d) and the aldoxane 2-(1-phenylethyl)-6-pentyl-5-methyl-5-phenyl-1,3-dioxan-4-ol.

IR (film): 3425 (s, broad), 2955 (s), 2931 (s), 2861 (m), 1725 (w), 1603 (w), 1465 (m), 1143 (m), 1113 (m) cm⁻¹.

MS: 375 (M⁺-1, 1), 305 (M⁺-71, 1), 217 (2), 188 (36), 176 (56), 134 (100), 118 (30), 43 (41).

### Example 3

Synthesis of Some Homo-aldols of Formula (V)

General Procedure

Typically, adipic acid (1-2 wt %) was added to an aldoxane obtained as described in Example 1 (2,6-diethyl-5-methyl-1,3-dioxane-4-ol was obtained according to Chuit et al. in Synthesis 1983, 294). The sample was then fractionally distilled in vacuo. Initially, the aldehyde liberated from the decomposing aldoxane was removed as the distillation flask was heated. Upon removal of the aldehyde, the aldol was obtained by distillation. Aldols were collected in receiving flasks cooled with a dry ice/acetone slurry to minimize dimerization. In this manner the aldols of propanal (a), butanal (b), isobutanal (c), pentanal (d) and hexanal (e) were isolated. Yields are based on amount of aldehyde used to make the crude aldoxanes. For the aldol of heptanal, 1 wt % Amberlite® IRC-50 (acidic ion exchange resin) was added to the aldoxane (3f) and the distillation conducted using a Kugelrohr distillation apparatus. Heptanal was removed at an oven temperature of 60° C. under vacuum (4 Pa) followed by distillation of the aldol at an oven temperature of 110° C. (3.3 Pa).

IR spectroscopy confirmed the aldol structure, showing a strong O—H absorption as well as a strong carbonyl absorption.

a. Propanal Aldol (3-hydroxy-2-methylpentanal): Yield=54%

B.p.:73-77° C./10 mbar

IR (film): 3430 (broad, s), 1722 (s) cm⁻¹.

b. Butanal Aldol (3-hydroxy-2-ethylhexanal): Yield=50%

B.p.: 78-83° C./7.3 Pa

IR (film): 3430 (broad, s), 1719 (s) cm⁻¹.

c. Isobutanal Aldol (3-hydroxy-2,2,4-trimethylpentanal): Yield=52%

B.p.: 86-87° C., 12 mbar

IR (film): 3478 (broad, s), 1718 (s) cm⁻¹.

¹H-NMR: 9.64 (1H, s), 3.54 (1H, d, J=4 Hz), 2,44 (1H, br s), 1.87 (1H, m), 1.13 (3H, s), 1.11 (3H, s), 0.96 (3H, d, J=6.7 Hz), 0.91 (3H, d, J=6.7 Hz);

¹³C-NMR: 207.0 (d), 80.3 (d), 50.6 (s), 30.0 (d), 21.7 (q), 19.8 (q), 18,7 (q), 17.3 (q).

d. Pentanal Aldol (3-hydroxy-2-propylheptanal): Yield=55%

B.p.: 72-76° C./6 Pa

IR (film): 3437 (broad, s), 1720 (s) cm⁻¹.

e. Hexanal Aldol (3-hydroxyl-2-butyloctanal): Yield=40%

B.p.: 103-104° C./2.6 Pa

IR (film): 3448 (broad, s), 1722 (s) cm⁻¹.

f. Heptanal Aldol (3-hydroxyl-2-pentylnonanal): Yield=53%

B.p.: 110° C./3.3 Pa (Kugelrohr distillation apparatus)

IR (film): 3448 (broad, s), 1721 (m) cm⁻¹.

### Example 4

Synthesis of Some Hetero-aldol of Formula (V)

a) 2,2-Dimethyl-3-hydroxypropanal

Isobutyraldehyde (100 g, 1.39 moles), paraformaldehyde (25 g, 0.83 moles) and water (50 ml) were added to a three-necked, round-bottomed flask equipped with over-head stirrer, thermometer and addition funnel. The mixture was cooled in a cold bath to 5° C. or less. An aqueous 10% KOH solution (25 ml) was added dropwise over a 90-minute period and then 200 ml of diethyl ether was added, the reaction mixture stirred 18 hours. Diethyl ether (200 ml) was added and the organic phase was extracted with water until the aqueous phase was neutral. The ether phase was dried over Na₂SO₄, filtered and concentrated on a rotary evaporator. Adipic acid (2 g) was added and the residue was distilled using a short path distillation head. Warm water (70° C.) was circulated through the distillation head condenser to prevent the distillate from crystallizing. 65 g of 2,2-dimethyl-3-hydroxypropanal (0.64 moles, 77% yield based on paraformaldehyde) was collected in a flasks cooled in a dry ice/acetone bath to prevent dimerization.

Bp: 65-70° C., 10 mbar

IR (film): 3425 (broad, s), 1727 (s), 1052 (s) cm⁻¹.

b) 2,2-Dimethyl-3-hydroxybutanal

Isobutyraldehyde (62 g, 0.86 moles), acetaldehyde (76 g, 1.72 moles) and diethyl ether (50 ml) were added to a three-necked round-bottomed flask equipped with a thermometer and addition funnel. The mixture was cooled in an ice bath to <5° C. An aqueous 10% KOH (30 ml) solution was added dropwise over a three-hour period and the reaction mixture stirred an additional two hours. Diethyl ether (200 ml) was added to the cold reaction mixture, the organic phase was extracted with water until the aqueous phase was neutral. The ether phase was dried over Na₂SO₄, filtered and concentrated on a rotary evaporator. Adipic acid (1.5 g) was added to the residue. The sample was fractionally distilled yielding 40.5 g (0.35 moles, 40.6% yield based on isobutyraldehyde) of 2,2-dimethyl-3-hydroxybutanal as a colorless liquid. The aldol was collected in receiving flasks cooled with a dry ice/acetone slurry to minimize dimerization.

B.p. 67-70° C., 15 mbar

IR(film): 3445 (broad, s), 1721 (s) cm⁻¹.

### Example 5

Synthesis of Aldoxanes from Aldols of Formula (V) and an Aldehyde of Formula (II)

6-Ethyl-2,5-dimethyl-1,3-dioxan-4-ol

Freshly distilled 3-hydroxy-2-methylpentanal (Example 3a, 20 g 0.172 moles) and 15 g (0.34 moles) of acetaldehyde were mixed at ambient for one day. Excess acetaldehyde was removed on a rotary evaporator. Fractional distillation of the remaining residue yielded 15.8 g of 6-ethyl-2,5-dimethyl-1,3-dioxan-4-ol (0.099 moles, 57% yield) as a mixture of at least three diastereomers.

Bp: 51-57° C., 2.6 Pa

IR (film): 3422 (s, broad), 1720 (w), 1149 (s), 1112 (s), 981 (m), 947 (m) cm⁻¹.

MS: 202 (M⁺, <1), 201 (M⁺-1, 4), 187 (3), 159 (5), 158 (5), 143 (M⁺-MeCO₂, 35), 100 (75), 99 (46), 70 (75), 43 (100).

General Procedure

Following the same type of procedure described hereinabove, the freshly distilled aldol, obtained such as described in examples 3 and 4, and the desired aldehyde R¹CHO were weighed into a flask and stirred for at least one day at ambient temperature (molar ratio used according to table 1 to 7). The composition of the reaction mixtures was assessed by GC-FID and GC-MS analysis of the acetate derivatives. The desired aldoxane was the major product formed in all the experiments listed. Minor components were the aldol dimer and unreacted aldehyde. Characterizing MS fragments (of the acetate derivative) are listed in the tables and the data are generally for the most abundant aldoxane isomer formed. The fragments ions listed are the molecular ion (M⁺), M⁺-1 ion, M⁺-R¹ (R¹ of formula I) and M⁺-59 (loss of acetate radical). Generally, the reaction mixtures were viscous liquids although several did crystallize (particularly those prepared with phenylacetaldehyde and Zestover). Aldoxanes prepared in this manner were used without further purification.

a) Aldoxanes from Homo-aldols of Formula (V)

Using the above general procedure, aldoxanes were synthesized from several perfumery aldehydes and propanal aldol (example 3a), see table 1; butanal aldol (example 3b), see table 2; isobutanal aldol (example 3c), see table 3; pentanal aldol (example 3d), see table 4; and hexanal aldol (example 3e), see table 5; and heptanal aldol (example 3f), see table 5a.

TABLE 1
Aldoxanes formed with propanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.1.1 | Isovaleraldehyde^{a)} | 2-methylpropyl | 244(<1) | 243(3) | 187(16) | 185(7) |
| 5.1.2 | Hexanal^{a)} | pentyl | 258(<1) | 257(4) | 187(20) | 199(4) |
| 5.1.3 | Octanal^{a)} | heptyl | 286(<1) | 285(1) | 187(14) | 227(3) |
| 5.1.4 | Decanal^{a)} | nonyl | 314(<1) | 313(4) | 187(47) | 255(7) |
| 5.1.5 | Phenylacetaldehyde^{a)} | benzyl | 278(0) | 277(1) | 187(51) | 219(5) |
| 5.1.6 | Trifernal ®^{b)} | 2-phenylpropyl | 306(1) | 305(<1) | 187(2) | 247(1) |
| 5.1.7 | 2,6-Dimethyl-5- | 1,5-dimethyl-4- | 298(1) | 297(<1) | 187(1) | 239(0) |
| | heptenal^{b)} | hexenyl | | | | |
| 5.1.8 | Zestover^{b)} | 2,4-dimethyl-3- | 296(<1) | 295(<1) | 187(1) | 237(1) |
| | | cyclohexenyl | | | | |
| 5.1.9 | 2-Methyldecanal^{b)} | 1-methylnonyl | 328(0) | 327(<1) | 187(11) | 269(<1) |

^{a)}molar ratio (aldol:aldehyde) = 1:1.2
^{b)}molar ratio (aldol:aldehyde) = 1:1
¹⁾the final aldoxanes are all 2-(R¹)-5-methyl-6-ethyl-1,3-dioxan-4-ol derivatives and in the above column it is specified only the name of the R¹ radical.

TABLE 2
Aldoxanes formed with butanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.2.1 | Hexanal^{a)} | pentyl | 286(<1) | 285(2) | 215(14) | 227(7) |
| 5.2.2 | Decanal^{b)} | nonyl | 342(<1) | 443(1) | 215(18) | 283(2) |
| 5.2.3 | Dodecanal^{b)} | undecyl | 370(<1) | 369(1) | 215(24) | 311(2) |
| 5.2.4 | Octanal^{a)} | heptyl | 314(<1) | 313(1) | 215(15) | 255(3) |
| 5.2.5 | Nonanal^{a)} | octyl | 328(<1) | 327(1) | 215(19) | 269(3) |
| 5.2.6 | Undecanal^{a)} | decyl | 356(<1) | 355(1) | 215(23) | 297(3) |
| 5.2.7 | 3,5,5-Trimethylhexanal^{a)} | 2,4,4- | 328(<1) | 327(1) | 215(17) | 269(2) |
| | | trimethylpentyl | | | | |
| 5.2.8 | 10-Undecenal^{a)} | 9-decenyl | 354(<1) | 353(1) | 215(18) | 295(3) |
| 5.2.9 | Citronellal^{a)} | 2,6-dimethyl-5- | 340(<1) | 339(1) | 215(2) | 281(0) |
| | | heptenyl | | | | |
| 5.2.10 | 2,6-Dimethyl-5-heptenal^{a)} | 1,5-dimethyl-4- | 326(3) | 325(1) | 215(2) | 261(<1) |
| | | hexenyl | | | | |
| 5.2.11 | Cyclosal^{c)} | 2-(4- | 376(1) | 375(<1) | 215(13) | 317(2) |
| | | isopropylphenyl)- | | | | |
| | | 1-methylethyl | | | | |
| 5.2.12 | Lilial ®^{c)} | 2-(4-tert- | 390(1) | 389(<1) | 215(16) | 331(2) |
| | | butylphenyl)-1- | | | | |
| | | methylethyl | | | | |
| 5.2.13 | Phenylacetaldehyde^{a)} | benzyl | 306(0) | 305(<1) | 215(34) | 247(4) |
| 5.2.14 | Trifernal ®^{b)} | 2-phenylpropyl | 334(2) | 333(<1) | 215(3) | 275(4) |
| 5.2.15 | Zestover^{a)} | 2,4-dimethyl-3- | 324(1) | 323(<1) | 215(2) | 265(2) |
| | | cyclohexenyl | | | | |

^{a)}molar ratio (aldol:aldehyde) = 1:1
^{b)}molar ratio (aldol:aldehyde) = 1:1.2
^{c)}molar ratio (aldol:aldehyde) = 1:0.9
¹⁾the final aldoxanes are all 2-(R¹)-5-ethyl-6-propyl-1,3-dioxan-4-ol derivatives and in the above column it is specified only the name of the R¹ radical.

TABLE 3
Aldoxanes formed with isobutanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.3.1 | Decanal^{a)} | nonyl | 342(<1) | 341(2) | 215(13) | 283(2) |
| 5.3.2 | Dodecanal^{b)} | undecyl | 370(<1) | 369(<1) | 215(8) | 311(1) |
| 5.3.3 | 2,6-Dimethyl-5 | 1,5-dimethyl-4- | 326(1) | 325(<1) | 215(1) | 267(<1) |
| | heptenal^{a)} | hexenyl | | | | |
| 5.3.4 | Phenylacetaldehyde^{a)} | benzyl | 306(0) | 305(<1) | 215(19) | 247(<1) |
| 5.3.5 | Cyclosal^{b)} | 2-(4- | 376(1) | 375(<1) | 215(7) | 317(1) |
| | | isopropylphenyl)- | | | | |
| | | 1-methylethyl | | | | |
| 5.3.6 | Lilial ®^{b)} | 2-(4-tert- | 390(1) | 389(<1) | 215(9) | 331(2) |
| | | butylphenyl)-1- | | | | |
| | | methylethyl | | | | |
| 5.3.7 | Trifernal ®^{a)} | 2-phenylpropyl | 334(1) | 333(<1) | 215(<1) | 275(2) |
| 5.3.8 | Heliopropanal^{a)} | 2-(1,3- | 378(11) | 377(<1) | 215(2) | 319(<1) |
| | | benzodioxol-5- | | | | |
| | | yl)-1- | | | | |
| | | methylethyl | | | | |
| 5.3.9 | Zestover^{a)} | 2,4-dimethyl-3- | 324(<1) | 323(<1) | 215(<1) | 265(<1) |
| | | cyclohexenyl | | | | |

^{a)}molar ratio (aldol:aldehyde) = 1:1.2
^{b)}molar ratio (aldol:aldehyde) = 1:1
¹⁾the final aldoxanes are all 2-(R¹)-5,5-dimethyl-6-isopropyl-1,3-dioxan-4-ol derivatives and in the above column it is specified only the name of the R¹ radical.

TABLE 4
Aldoxanes formed with pentanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.4.1 | Octanal^{a)} | heptyl | 342(<1) | 341(1) | 243(5) | 283(2) |
| 5.4.2 | Decanal^{b)} | nonyl | 370(0) | 369(<1) | 243(2) | 311(<1) |
| 5.4.3 | Undecanal^{a)} | decyl | 384(<1) | 383(1) | 243(9) | 325(2) |
| 5.4.4 | Dodecanal^{b)} | undecyl | 398(<1) | 397(1) | 243(7) | 339(2) |
| 5.4.5 | 2-Methylundecanal^{b)} | 1-methyldecyl | 398(<1) | 397(1) | 243(18) | 339(2) |
| 5.4.6 | 3,5,5-Trimethylhexanal^{a)} | 2,4,4- | 356(0) | 355(1) | 243(9) | 297(3) |
| | | trimethylpentyl | | | | |
| 5.4.7 | Liminal ®^{c)} | (4R)-1-p- | 380(0) | 379(<1) | 243(<1) | 321(<1) |
| | | menthen-9-yl | | | | |
| 5.4.8 | Interleven ® Aldehyde^{b)} | 9-or 8-decenyl | 382(0) | 381(0) | 243(1) | 323(<1) |
| 5.4.9 | 9-Undecenal^{c)} | 8-decenyl | 382(0) | 381(0) | 243(1) | 323(0) |
| 5.4.10 | 2,6-Dimethyl-5- | 1,5-dimethyl-4- | 354(1) | 253(<1) | 243(1) | 295(<1) |
| | heptenal^{c)} | hexenyl | | | | |
| 5.4.11 | Citronellal^{a)} | 2,6-dimethyl-5- | 368(<1) | 367(1) | 243(<1) | 309(<1) |
| | | heptenyl | | | | |
| 5.4.12 | Phenylacetaldehyde^{a)} | benzyl | 334(0) | 333(<1) | 243(16) | 275(2) |
| 5.4.13 | Cyclosal^{d)} | 2-(4-iso- | 404(<1) | 403(0) | 243(6) | 345(3) |
| | | propylphenyl)-1- | | | | |
| | | methylethyl | | | | |
| 5.4.14 | Lilial ®^{d)} | 2-(4-tert- | 418(<1) | 417(<1) | 243(9) | 359(<1) |
| | | butylphenyl)-1- | | | | |
| | | methylethyl | | | | |
| 5.4.15 | Heliopropanal^{b)} | 2-(1,3- | 406(2) | 405(0) | 243(0) | 347(0) |
| | | benzodioxol-5- | | | | |
| | | yl)-1- | | | | |
| | | methylethyl | | | | |
| 5.4.16 | Trifernal ®^{d)} | 2-phenylpropyl | 362(<1) | 361(<1) | 243(1) | 303(2) |
| 5.4.17 | Zestover^{c)} | 2,4-dimethyl-3- | 352(<1) | 351(<1) | 243(1) | 293(1) |
| | | cyclohexenyl | | | | |

^{a)}molar ratio (aldol:aldehyde) = 1:1.1
^{b)}molar ratio (aldol:aldehyde) = 1:1.2
^{c)}molar ratio (aldol:aldehyde) = 1:1
^{d)}molar ratio (aldol:aldehyde) = 1:0.9
¹⁾the final aldoxanes are all 2-(R¹)-5-propyl-6-butyl-1,3-dioxan-4-ol derivatives and in the above column it is specified only the name of the R¹ radical.

TABLE 5
Aldoxanes formed with hexanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.5.1 | Propanal^{a)} | ethyl | 300(<1) | 299(<1) | 271(2) | 241(2) |
| 5.5.2 | Octanal^{b)} | heptyl | 370(<1) | 369(<1) | 271(5) | 311(1) |
| 5.5.3 | Decanal^{b)} | nonyl | 398(<1) | 397(<1) | 271(7) | 339(1) |
| 5.5.4 | Dodecanal^{b)} | undecyl | 426(0) | 425(<1) | 271(9) | 367(<1) |
| 5.5.5 | Trifernal ®^{a)} | 2-phenylpropyl | 390(1) | 389(<1) | 271(1) | 331(2) |
| 5.5.6 | Phenylacetaldehyde^{c)} | benzyl | 362(0) | 361(<1) | 271(17) | 303(1) |
| 5.5.7 | Bourgeonal ®^{b)} | 2-(4-tert- | 432(<1) | 431(<1) | 271(<1) | 373(1) |
| | | butylphenyl)ethyl | | | | |
| 5.5.8 | Citronellal^{b)} | 2,6-dimethyl-5- | 396(<1) | 395(<1) | 271(<1) | 337(1) |
| | | heptenyl | | | | |
| 5.5.9 | 2,6-Dimethyl-5- | 1,5-dimethyl-4- | 382(2) | 381(1) | 271(1) | 323(1) |
| | heptenal^{b)} | hexenyl | | | | |
| 5.5.10 | 9-Undecenal^{b)} | 8-undecenyl | 410(<1) | 409(1) | 271(5) | 351(2) |
| 5.5.11 | 2-Methylundecanal^{b)} | 1-methyldecyl | 426(<1) | 425(1) | 271(17) | 367(3) |
| 5.5.12 | Hydratropic | 1-phenylethyl | 276(<1) | 275(,1) | 271(25) | 317(4) |
| | aldehyde^{b)} | | | | | |
| 5.5.13 | Zestover^{b)} | 2,4-dimethyl-3- | 380(1) | 379(<1) | 271(7) | 321(4) |
| | | cyclohexenyl | | | | |

^{a)}molar ratio (aldol:aldehyde) = 1:0.9
^{b)}molar ratio (aldol:aldehyde) = 1:1
^{c)}molar ratio (aldol:aldehyde) = 1:1.1
¹⁾the final aldoxanes are all 2-(R¹)-5-butyl-6-pentyl-1,3-dioxan-4-ol derivatives and in the above column it is specified only the name of the R¹ radical.

TABLE 5A
Aldoxanes formed with heptanal aldol

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.5a.1 | Citronellal^{a)} | 2,6-dimethyl-5- | 424(<1) | 423(<1) | 299(<1) | 365(<1) |
| | | heptenyl | | | | |
| 5.5a.2 | 9-Undecenal^{a)} | 8-undecenyl | 438(0) | 437(<1) | 299(3) | 379(1) |
| 5.5a.3 | 2-Methylundecanal^{a)} | 1-methyldecyl | 454(0) | 453(<1) | 299(9) | 395(1) |
| 5.5a.4 | Zestover^{a)} | 2,4-dimethyl-3- | 408(<1) | 407(0) | 299(1) | 349(2) |
| | | cyclohexenyl | | | | |

^{a)}molar ratio (aldol:aldehyde) = 1:1
¹⁾the final aldoxanes are all 2-(R¹)-5-pentyl-6-hexyl-1,3-dioxan-4-ol derivatives and in the above column it is specified only the name of the R¹ radical.
^{b)}Aldoxanes from hetero-aldols of formula (V)
Using the above general procedure it was synthesized the aldoxanes from several perfumery aldehydes and 2,2-dimethyl-3-hydroxybutanal (example 4b), see table 6.

TABLE 6
Aldoxanes formed with 2,2-dimethyl-3-hydroxybutanal

| | | | MS Fragment Ions, m/z | | | |
|---|---|---|---|---|---|---|
| | Aldehyde | Name of the | (relative abundance) | | | |
| N° | R¹CHO | Final aldoxane¹⁾ | M⁺ | M⁺-1 | M⁺-R¹ | M⁺-59 |
| 5.6.1 | Decanal^{a)} | nonyl | 314(<1) | 313(2) | 187(26) | 255(1) |
| 5.6.2 | Citronellal^{a)} | 2,6-dimethyl-5- | 312(1) | 311(1) | 187(3) | 253(0) |
| | | heptenyl | | | | |
| 5.6.3 | Phenylacetaldehyde^{a)} | benzyl | 278(<1) | 277(1) | 187(63) | 219(5) |
| 5.6.4 | 3-Phenylpropanal^{a)} | 2-phenylethyl | 292(5) | 291(1) | 187(4) | 233(4) |

^{a)}molar ratio (aldol:aldehyde) = 1:1
¹⁾the final aldoxanes are all 2-(R¹)-5,5-dimethyl-6-methyl-1,3-dioxan-4-ol derivatives and in the above column it is specified only the name of the R¹ radical.

### Example 6

Thermal-release of Aldehydes

a) Thermolysis of 2,6-dibuty-5-pentyl-1,3-dioxan-4-ol

In a typical experiment 8.7 g of pentanal aldol (example 3d) (0.05 moles) and 3.4 g of pentanal (0.04 moles) were mixed into a three-necked 100 ml flask with magnetic stir bar and gas outlet. The mixture was stirred at least two days to allow formation of the aldoxane. The total weight of the vessel and mixture was recorded. The gas outlet was connected to a series of three cold traps cooled with dry ice/acetone slurries. The mixture then was placed in an oil bath at the desired temperature and well stirred. Nitrogen was bubbled into the mixture (280 ml/min) through a needle. Periodically the vessel was removed from the oil bath and weighed. The weight difference was divided by the mass of pentanal added to determine percentage of pentanal released (i.e. a loss of 3.4 g=100% pentanal released, see Table 7). GC-MS analysis of the material collected in the cold traps confirmed that the latter was composed almost exclusively of pentanal. Minor materials detected were pentanoic acid, 2-propyl-2-heptenal and the pentanal aldol. At the end of the measurements, the residue in the flask was set at room temperature for one day and then analyzed by GC-MS as the acetate derivative and found to be the aldol dimer with only a trace of the original aldoxane present.

TABLE 7
Percentage of pentanal released from the aldoxane
obtained by the reaction between the pentanal aldol and pentanal

| Time (hrs) | 80° C.¹⁾ | 60° C.¹⁾ | 40° C. | 22° C. |
|---|---|---|---|---|
| 1 | 76 | 21 | 6 | |
| 2 | 93 | 27 | 9 | |
| 3 | 99 | 30 | | |
| 4 | | 48 | 16 | 5 |
| 5 | | 50 | | |
| 6 | | 59 | 19 | |
| 7 | | 64 | | |
| 8 | | 69 | | 8 |
| 9 | | 72 | 21 | |
| 24 | | | 43 | 13 |
| 48 | | | | 16 |

¹⁾average of the results obtained on two separated experiments

Ten grams of the compound described in Example 2b were added to a three-necked, 100 ml flask and heated at 80° C. as described above in example 6a. After seven hours, 2.4 grams of material had distilled from the flask. IR and GC-MS analysis showed this material was hexanal with only a small amount of hydratropic aldehyde (hexanal:hydratopic aldehyde GC-FID area ratio 99.4:0.6). In a second experiment, 10 grams of the aldoxane was treated in the same manner for 24 hours. Four grams of volatile material was distilled from the flask. IR and GC-MS analysis showed this material was composed predominantly of hexanal and hydratropic aldehyde (GC-FID area ratio 87:13, respectively).

### Example 7

Test on Cloth

Portions of unfragranced Downy liquid fabric softener (Procter & Gamble, Cincinnati, Ohio) were dosed with either 0.1% (w/w) of the selected aldehyde or an amount of the corresponding aldoxane, equivalent to 0.1% aldehyde, and obtained as described in Example 5 (e.g., 2-heptyl-5-propyl-6-butyl-1,3-dioxan-4-ol, example 5 N° 5.4.1, was used at 0.22% in the fabric softener). Cotton terry wash clothes and towels (weight about 2 kg) were washed in a top-loading washing machine using an unfragranced, liquid laundry detergent (Tide Free, Procter & Gamble). A hot water wash with cold water rinse was used. 35 grams of the fabric softener was placed in the liquid fabric conditioner dispenser in each washing machine at the start of the wash. At the end of the wash, the towels were dried in a tumble drier (60 minutes). The dry towels were wrapped in aluminum foil for storage at room temperature prior to evaluations. Pair-difference tests were performed comparing towels treated with an aldoxane against towels treated with the corresponding free aldehyde. Panelists were asked to choose the sample with the stronger odor. Statistical analysis was done using a two-tailed paired comparison test. Results are summarized in Table 8.

TABLE 8
Test between aldoxane-treated and aldehyde-treated
fabric for odor strength

| | | | | Panelist Choosing |
|---|---|---|---|---|
| Aldoxane | Level in Fabric | Days after | Number of | Aldoxane-Treated |
| N°^{a)} | Softener^{b)} | Laundering | Panelist | Fabric as Stronger |
| 5.4.1 | 0.22% | 2 | 6 | 6 |
| 5.4.4 | 0.18% | 3 | 6 | 6 |
| 5.4.4 | 0.18% | 9 | 30 | 27 |
| 5.4.10 | 0.22% | 2 | 6 | 5 |
| 5.4.10 | 0.22% | 8 | 30 | 21 |
| 5.4.11 | 0.20% | 3 | 6 | 6 |
| 5.4.11^{c)} | 0.20% | 2 | 30 | 27 |
| 5.4.11 | 0.20% | 9 | 30 | 21 |
| 5.4.18 | 0.20% | 1 | 6 | 6 |
| 5.4.18 | 0.20% | 2 | 30 | 20 |
| 5.4.18 | 0.20% | 7 | 30 | 21 |
| 5.4.14 | 0.19% | 1 | 6 | 6 |

^{a)}aldoxane N°: corresponds to the same numbering as in example 5.
^{b)}weight percentage; equivalent to 0.1% of free aldehyde
^{c)}fabric softener one-month old.

### Example 8

Test on Cloth

A) Two liquid fabric softener samples were prepared using an unfragranced fabric softener containing either 0.2% (w/w) of Zestover or 0.45% of 2-(2,4-dimethyl-3-cyclohexenyl)-5-propyl-6-butyl-1,3-dioxan-4-ol (Zestover precursor, example 5 N°5.4.17). The formula of the fabric softener was: 16.7% Stepantex VS 90 (Stepan Company, Northfield, Ill., USA), 0.2% of a 10% aqueous CaCl₂ solution, 0.3% colorant (1% aqueous solution of Blue Sandolan, origin: Clariant), 82.8% deionized water, percentage being relative to the total weight.

Thirty-five small cotton towels (46×38 cm, load weight about 2.5 kg) were washed in a top-loading washing machine using an unfragranced, liquid laundry detergent (Tide Free, Procter & Gamble). A warm-water wash with cold-water rinse was used. 30 grams of fabric softener were added to the washing machine as it was filling for the final cold-water rinse. The fabric was tumble-dried for about 43 minutes. Each towel was placed in an individual plastic bin with a lid.

Both the aldoxane-treated and aldehyde-treated fabric were evaluated for odor strength on a 9-point scale (1=no odor; 9=extremely strong). Each sample was evaluated daily for seven days after washing by a panel of the same forty-four individuals. The first evaluation was done the day of washing (day 0). The data obtained for each day was merged and the mean odor strength ratings compared using t-tests.

The aldoxane-treated fabric was significantly stronger than the aldehyde-treated fabric on each day of the evaluation, at a confidence interval of 99.8% or greater. The odor strength rating for each sample over the seven days is presented in Table 9.

TABLE 9
Odor Strength Rating of Aldehyde-Treated vs.
Aldoxane-Treated Cotton Fabric

| | |
|---|---|
| Number of | Mean Strength Rating (Std. Dev.) |

| Day | evaluations | aldehyde-treated | aldoxane-treated |
|---|---|---|---|
| 0 | 130 | 2.03(1.21) | 4.26(2.00) |
| 1 | 86 | 2.10(1.37) | 3.66(1.77) |
| 3 | 82 | 1.93(1.17) | 3.56(1.93) |
| 4 | 84 | 1.89(1.18) | 3.11(1.95) |
| 5 | 84 | 1.70(1.00) | 3.23(1.67) |
| 6 | 82 | 1.69(1.07) | 3.05(1.68) |
| 7 | 127 | 1.81(1.12) | 2.91(1.65) |

(value in brackets represent the standard deviation)

B) When, instead of the above mentioned aldoxane and corresponding perfuming aldehyde were used 0.45% w/w 2-methylundecanal aldoxane (see example 5, N°5.5.11) and 0.2% w/w 2-methylundecanal, the results obtained were as reported in table 10:

TABLE 10
Odor Strength Rating of Aldehyde-Treated vs.
Aldoxane-Treated Cotton Fabric

| | |
|---|---|
| Number of | Mean Strength Rating (Std. Dev.) |

| Day | evaluations | aldehyde-treated | aldoxane-treated |
|---|---|---|---|
| 0 | 24 | 3.17(1.83) | 4.5(2.04) |
| 3 | 23 | 3.04(1.97) | 5.0(2.10) |
| 7 | 24 | 3.13(1.96) | 4.17(2.01) |

(value in brackets represent the standard deviation)

C) When, instead of the above mentioned aldoxane and corresponding perfuming aldehyde were used 0.45% w/w 9-undecenal aldoxane (see example 5, N°5.4.9) and 0.2% w/w 9-undecenal, the results obtained were as reported in table 11:

TABLE 11
Odor Strength Rating of Aldehyde-Treated vs.
Aldoxane-Treated Cotton Fabric

| | |
|---|---|
| Number of | Mean Strength Rating (Std. Dev.) |

| Day | evaluations | aldehyde-treated | aldoxane-treated |
|---|---|---|---|
| 0 | 23 | 3.35(1.85) | 2.61(1.50) |
| 3 | 25 | 2.76(1.51) | 3.84(1.84) |
| 7 | 25 | 2.32(1.41) | 3.40(1.83) |

(value in brackets represent the standard deviation)

D) When, instead of the above mentioned aldoxane and corresponding perfuming aldehyde were used 0.45% w/w aged Zestover-aldoxane (see, example 5 N°5.4.17) and 0.2% w/w aged Zestover (“aged” means that liquid fabric softeners containing either Zestover aldoxane or Zestover were stored for 8 weeks at 45° C.), the results obtained were as reported in table 12:

Table 12: Odor Strength Rating of Aldehyde-Treated vs. Aldoxane-Treated Cotton Fabric

| | |
|---|---|
| Number of | Mean Strength Rating (Std. Dev.) |

| Day | evaluations | aldehyde-treated | aldoxane-treated |
|---|---|---|---|
| 0 | 134 | 1.91(1.16) | 2.50(1.57) |
| 1 | 89 | 1.96(1.26) | 2.62(1.50) |
| 2 | 88 | 2.00(1.07) | 2.40(1.25) |
| 3 | 90 | 1.94(0.92) | 2.30(1.17) |
| 4 | 86 | 2.03(1.23) | 2.33(1.27) |
| 5 | 87 | 1.74(0.90) | 2.31(1.37) |
| 6 | 85 | 1.93(1.33) | 2.19(1.39) |
| 7 | 130 | 1.97(1.13) | 2.12(1.37) |

(value in brackets represent the standard deviation)

### Example 9

Dryer sheets were prepared according to methods known in the art containing either 2.2% MNA aldoxane (example 5 N°5.5.10) or a mixture of 1% MNA in dryer sheet base; namely, DXP 3505 002C Ester Quat made by Goldschmidt. The base containing aldoxane or aldehyde was then coated onto dryer sheets (1.7 g base per 0.7 g of dryer sheet cut into 16×139 cm sheets).

Thirty-five small cotton towels (46×38 cm, load weight about 2.5 kg) were washed in a top-loading washing machine using an unfragranced, liquid laundry detergent (Tide Free, Procter & Gamble). A warm water wash with cold water rinse was used. The fabric was then tumble-dried for about 43 minutes with the above respective fabric-softening sheet. Each towel was placed in an individual plastic bin with a lid.

Both the aldoxane-treated and aldehyde-treated fabric were evaluated for odor strength by a panel of 15 individuals. 14 of the panelist chosen the aldoxane-treated towels as being the strongest. This finding is statistically significant at a 99.9% confidence limit.

1. A composition of matter comprising at least one compound of formula 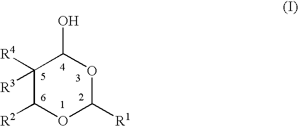
wherein R¹ represents an organic residue CY₃ of a perfuming or flavoring aldehyde of formula Y₃CCHO, Y being a hydrogen atom, a C₁ to C₂₀ linear, branched, cyclic or poly-cyclic saturated, unsaturated, aromatic or alkylaryl hydrocarbon radical, with the hydrocarbon radical optionally comprising up to three oxygen or nitrogen atoms and being optionally substituted; and two Y groups optionally bonded together to form a saturated, unsaturated or aromatic ring having 5 to 20 carbon atoms, with the ring optionally being substituted;
R² represents a R¹ group, a Y group or a C₅ to C₁₀ aromatic ring, with the ring optionally up to three oxygen or nitrogen atoms and optionally being substituted; and
R³ and R⁴ represent each a Y group or are bonded together to form a saturated or unsaturated ring having 5 to 20 carbon atoms, with the ring optionally being substituted; and
a perfume or flavor base; provided that muscat wine extracts are excluded.

2. The composition of matter according to claim 1, wherein the active ingredient of formula (I) includes
R¹, R² and Y as defined as in claim 1;
R³ representing a C₁ to C₁₆ linear, branched, cyclic or poly-cyclic saturated, unsaturated, aromatic or alkylaryl hydrocarbon radical, with the hydrocarbon radical optionally comprising up to three oxygen or nitrogen atoms and optionally being substituted; and
R⁴ represents a Y group or R⁴ and R³ are bonded together to form a saturated or unsaturated ring having 5 to 15 carbon atoms, the ring optionally being substituted.

3. The composition of matter according to claim 1, wherein the active ingredient of formula (I) includes
R¹ representing an organic residue derived from an active aldehyde of formula R¹CHO selected from the group consisting of hydroxycitronellal, citronellal, 3-(4-methoxyphenyl)-2-methylpropanal, the linear C₈ to C₁₂ alkyl aldehydes, 3-(4-isopropylphenyl)-2-methylpropanal, 3-(4-tert-butylphenyl)-2-methylpropanal, 4- and 3-(4-hydroxy-4-methylpentyl)-3-cyclohexene-1-carbaldehyde, 3-(4-tert-butylphenyl)propanal, 3-(1,3-benzodioxol-5-yl)-2-methylpropanal, 2,4-dimethyl-3-cyclohexene-1-carbaldehyde, 3- and 4-(3,3-dimethyl-5-indanyl)propanal, 8(9)-methoxy-tricyclo[5.2.1.0.(2,6)]decane-3(4)-carbaldehyde and 3-phenylbutanal;
R² representing a R¹ group, a hydrogen atom or a C₁ to C₁₆ linear, branched, cyclic or polycyclic alkyl, alkenyl or alkylaryl hydrocarbon radical, with the hydrocarbon radical optionally being substituted, or a C₅ to C₆ aromatic ring, optionally comprising up to three oxygen or nitrogen atoms and optionally being substituted;
R³ represents a C₁ to C₁₆ linear, branched, cyclic or poly-cyclic saturated, unsaturated, aromatic or alkylaryl hydrocarbon radical, with the hydrocarbon radical optionally comprising up to three oxygen or nitrogen atoms and optionally being substituted; and
R⁴ represents a hydrogen atom or a R³ group; or R⁴ and R³ are bonded together to form a saturated or unsaturated ring having 5 to 10 carbon atoms, with the ring optionally being substituted.

4. The composition of matter according to claim 1, wherein the active ingredient of formula (I) includes
R¹ representing an organic residue derived from an active aldehyde of formula R¹CHO selected from the group consisting of hydroxycitronellal, citronellal, 3-(4-methoxyphenyl)-2-methylpropanal, the linear C₈ to C₁₂ alkyl aldehydes, 3-(4-isopropylphenyl)-2-methylpropanal, 3-(4-tert-butylphenyl)-2-methylpropanal, 4- and 3-(4-hydroxy-4-methylpentyl)-3 -cyclohexene-1 -carbaldehyde, 3-(4-tert-butylphenyl)propanal, 3-(1,3-benzodioxol-5-yl)-2-methylpropanal, 2,4-dimethyl-3-cyclohexene-1-carbaldehyde, 3- and 4-(3,3-dimethyl-5-indanyl)propanal, 8(9)-methoxy-tricyclo[5.2.1.0.(2,6)]decane-3(4)-carbaldehyde and 3-phenylbutanal; and
R² and R³ represent a C₃ to C₁₀ linear, branched, cyclic or polycyclic saturated, unsaturated, aromatic or alkylaryl hydrocarbon radical, and R⁴ represents a hydrogen atom or a methyl or ethyl group.

5. The composition of matter according to claim 1, wherein the active ingredient of formula (I) is susceptible of being obtained by a reaction comprising the following step:
a) mixing together, the three aldehydes of the formulae (II), (III) and (IV), 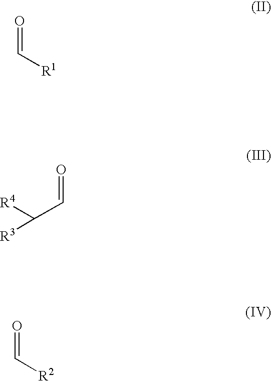
wherein R¹, R², R³ and R⁴ have the meaning as defined in formula (I), in the presence of a base, such as an alkaline hydroxide or C₁ to C₄ alkoxide, and a temperature comprised between −10° C. and +10° C., preferably between 0° C. and 5° C., and aldehydes (III) and (IV) being in at least an equimolar amount in respect of aldehyde (II); or
b) reacting, at a temperature comprised between −10° C. and 50° C., preferably between 0° C. and 30° C., an active aldehyde (II), as defined hereinabove, with an aldol of formula 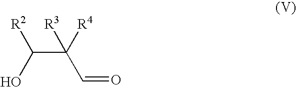
wherein R², R³ and R⁴ have the meaning as defined in formula (I), the latter being susceptible of being obtainable by an aldol reaction between an aldehyde of formula (III) and an aldehyde of formula (IV).

6. A compound of formula (I) as defined in claim 1, provided that 5-methyl-2,6-bis(1-methylethyl)-1,3-dioxan-4-ol, 2,6-diethyl-5-methyl-1,3-dioxan-4-ol, 2,6-dimethyl-1,3-dioxan-4-ol, 6-hexyl-2-(1-methylethyl)-1,3-dioxan-4-ol, 5,5,6-trimethyl-2-(1-methylethyl)-1,3-dioxan-4-ol, 5,5-dimethyl-2,6-bis(1-methylethyl)-1,3-dioxan-4-ol, 2,6-dibenzyl-5-phenyl-1,3-dioxan-4-ol, 2-ethyl-6-methyl-1,3-dioxan-4-ol, 2-(1-methylethenyl)-1,3-dioxan-4-ol, 6-hexyl-2-(2-octanol-1-yl)-1,3-dioxan-4-ol and 5-ethyl-2,6-dipropyl-1,3-dioxan-4-ol are excluded.

7. A composition of matter comprising at least one compound of formula (I), as defined in claim 1, and at least one perfumery or flavor carrier.

8. A perfumed article comprising:
i) at least one compound of formula (I), as defined in claim 1; and
ii) a consumer product base.

9. The perfumed article according to claim 8, wherein the consumer product base is in the form of a solid or liquid detergent, a fabric softener, a perfume, a cologne, an after-shave lotion, a perfumed soap, a shower or bath salt, mousse, oil or gel, a hygiene product, a hair care product, a shampoo, a body-care product, a deodorant, a antiperspirant, an air freshener, a cosmetic preparation, a fabric refresher, an ironing water, a paper, a wipe or a bleach.

10. A perfumed article comprising:
i) the composition as defined in claim 7; and
ii) a consumer product base.

11. The perfumed article according to claim 10, wherein the consumer product base is in the form of a solid or liquid detergent, a fabric softener, a perfume, a cologne, an after-shave lotion, a perfumed soap, a shower or bath salt, mousse, oil or gel, a hygiene product, a hair care product, a shampoo, a body-care product, a deodorant, a antiperspirant, an air freshener, a cosmetic preparation, a fabric refresher, an ironing water, a paper, a wipe or a bleach.

12. A flavored article comprising:
i) at least one compound of formula (I), as defined in claim 1; and
ii) a foodstuff base.

13. The flavored article according to claim 12, wherein the foodstuff base is in the form of a dry powder or concentrated composition for instant beverage, a chewing gum or a baking application.

14. A flavored article comprising:
i) a composition as defined in claim 7; and
ii) a foodstuff base.

15. The flavored article according to claim 14, wherein the foodstuff base is in the form of a dry powder or concentrated composition for instant beverage, a chewing gum or a baking application.

16. A process for the perfuming of a surface or a process for intensifying, prolonging or deferring the diffusion effect of the characteristic fragrance of a fragrant aldehyde on a surface, which comprises treating the surface with at least one compound of formula (I) 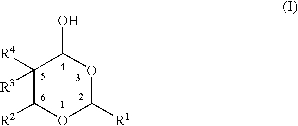
wherein R¹ represents an organic residue CY₃ of a perfuming or flavoring aldehyde of formula Y₃CCHO, Y being a hydrogen atom, a C₁ to C₂₀ linear, branched, cyclic or poly-cyclic saturated, unsaturated, aromatic or alkylaryl hydrocarbon radical, with the hydrocarbon radical optionally comprising up to three oxygen or nitrogen atoms and being optionally substituted; and two Y groups optionally bonded together to form a saturated, unsaturated or aromatic ring having 5 to 20 carbon atoms, with the ring optionally being substituted;
R² represents a R¹ group, a Y group or a C₅ to C₁₀ aromatic ring, with the ring optionally up to three oxygen or nitrogen atoms and optionally being substituted; and
R³ and R⁴ represent each a Y group or are bonded together to form a saturated or unsaturated ring having 5 to 20 carbon atoms, with the ring optionally being substituted;
wherein the compound(s) is present in a fragrance effective amount.

17. A method for perfuming a surface or object which comprises adding a composition according to claim 1, which includes a perfume base comprising an active aldehyde, to a chemically aggressive medium, and then releasing the active aldehyde at the desired moment, wherein the composition protects the aldehyde from degradation due to exposure to the chemically agressive medium.

18. A method for flavoring an edible substance which comprises adding a composition according to claim 1, which includes a flavor base comprising an active aldehyde, to a chemically aggressive medium, and then releasing the active aldehyde at the desired moment, wherein the composition protects the aldehyde from degradation due to exposure to the chemically agressive medium.
